(19) United States
(12) Patent Application Publication
(10) Pub. No.: US 2018/0243388 A1
(43) Pub. Date: Aug. 30, 2018

(54) COMPOSITIONS AND METHODS FOR TREATING MELANOMA

(71) Applicant: Cyvax, Inc., Atlanta, GA (US)

(72) Inventors: Richard Markham, Columbia, MD (US); James Gordy, Baltimore, MD (US)

(73) Assignee: Cyvax, Inc., Atlanta, GA (US)

(21) Appl. No.: 15/526,256

(22) PCT Filed: Nov. 12, 2015
(86) PCT No.: PCT/US15/60371

§ 371 Date: May 11, 2017

Related U.S. Application Data
(60) Provisional application No. 62/078,225, filed on Nov. 11, 2014.

(51) Int. Cl. A61K 39/00; A61K 38/19; A61K 38/20; A61P 35/00

(52) U.S. Cl. CPC A61K 39/0011; A61P 35/00; A61K 38/2066; A61K 38/195

(57) ABSTRACT

Melanoma vaccines are provided. Compositions and methods are provided for making and using melanoma vaccine constructs, alone, or in combination with at least one adjuvant. Compositions and methods can be in combination with other therapeutic compositions. The melanoma vaccine constructs of DNA or protein.

### SEQUENCE LISTING

The instant application contains a Sequence Listing which has been submitted electronically in ASCII format and is hereby incorporated by reference in its entirety. Said ASCII copy, created on Nov. 12, 2015, is named 19540_0104 PTWO_SL.txt and is 11,000 bytes in size.

### TECHNICAL FIELD

This invention relates to the fields of cancer treatment and vaccine mediated therapies.

### BACKGROUND OF THE INVENTION

Of the seven most common cancers in the US, melanoma is the only one to have increasing incidence in the 20 century, with an estimated 76,100 incident cases of invasive melanoma diagnosed in the US in 2014, and 9,710 estimated deaths. Additionally, melanoma is very aggressive. According to the American Cancer Society, once the cancer progresses to stage IIIB, the ten-year survival rate dips below 50%, with that rate plummeting to 10-15% for stage IV. Although melanoma is highly immunogenic, the anti-inflammatory tumor microenvironment greatly inhibits any immune-based intervention. The discovery of immune checkpoint inhibitors targeting tumor microenvironment interactions with effector T-cells has marked a major discovery and paradigm shift in research and treatment modalities. The success of ipilimumab (anti-cytotoxic T-lymphocyte-associated antigen 4 [αCTLA-4]) in the clinic has paved the way for competing anti-Programmed Cell Death 1 (αPD-1) drugs pembrolizumab and nivolumab. These antibodies directly target proteins on the surface of effector T cells linked to signaling pathways that inhibit T cell activation. Melanoma generates a tolerogenic environment that is active at stages that precede generation of effector T cells.

Another promising line of investigation is that of cancer vaccines. A novel DNA vaccine platform has been developed that includes the chemokine macrophage inflammatory protein 3α (MIP3α/CCL20) fused to the melanoma-associated antigen GP100. This platform is superior to standard DNA vaccines because the chemokine targets nascent protein to the immature dendritic cells (iDCs) that are pertinent to the development of an adaptive immune response. The iDCs process antigen via both class I and class II pathways, jump-starting both humoral and cell-mediated immunity. Studies in a malaria challenge system have demonstrated that combining this iDC targeting vaccine construct with an adjuvant results in resistance to infection that is improved by orders of magnitude, compared to either adjuvant or vaccine construct alone. The aspects of the malaria challenge system were presented in U.S. Pat. No. 8,557,248, which is herein incorporated by reference. This vaccine platform has also been shown to work in malaria with circumsporozoite protein antigen, in melanoma prophylactically with gp100 antigen, and in lymphoma therapeutically with OFA-iLRP antigen. Preliminary data show that this construct is efficacious in a therapeutic setting with melanoma, prolonging median survival by 29%.

Previous melanoma therapies have provided hope for better treatments but other treatments have failed because later stage melanomas eventually escape the effect of the treatment. This failure can be attributable to mutations arising in the proteins targeted by the immune response or by mechanisms of immune tolerance that downregulate the immune response to tumor antigens, which are frequently proteins normally expressed, albeit at lower frequency, on non-malignant cells. While some current therapies seek to counteract some of the tolerance mechanisms, only a minority of patients respond to such interventions.

There is a need for the development of, and improvement of vaccines for the treatment of melanoma.

### SUMMARY

The invention provides compositions and methods for treatment of various cancers. Vaccine constructs are provided comprising a cytokine fused to a cancer antigen. The present invention provides DNA and protein vaccine constructs, which can be based on the fusion of a cytokine, e.g., MIP-3α, and a melanoma-associated antigen, e.g., GP100. The vaccine constructs of the present invention can be provided for use in combination with various adjuvants, and various other cancer therapies. The present invention provides for combining the vaccine constructs with anti-IL-10, for example.

The present invention also provides methods of making and using the DNA and protein vaccine constructs in the treatment of melanoma. Also provided are methods for using the protein vaccine constructs in Antibody-Coupled T-cell Receptor (“ACTR”) technology.

### BRIEF DESCRIPTION OF THE DRAWINGS

FIG. 1A: Schematic of vaccine plasmid expressed insert. L=leader sequence.

FIG. 1B: Western blot against myc tag of 293T cell lysates 48 hours after Lipofectamine plasmid transfection. ME4.2 and ME6 are two independent DNA extraction preps. N21-4 is a control construct with a mutated MIP-3α. (+) is purified myc-tagged CSP protein. (−) control untransfected 293T cells. Below primary blot is β-actin protein loading control blot.

FIG. 2A: Experimental schedule. N=6. Challenged with 1×105 B16 cells.

FIG. 2B: Survival analysis. Significance assessed by log-rank test. Endpoints consisted of mouse death, a tumor dimension exceeding 2 cm, or excessive tumor ulceration and bleeding.

FIG. 3A: ELISpot assays, n=4. APC's loaded with negative hemagglutinin (HA) or gp100.

FIG. 3B: In Cell ELISA assays, n=15. ELISA plate coated with permeabilized, fixed B16 cells and covered with mouse serum at 1:100 dilutions and flourescent reaction incubated at room temperature for one hour before measuring at 405 nm. Analyzed significance by student's T-test.

FIG. 4A: Experimental schedule OF N=7-8. Challenged with 5×104 B16 cells.

FIG. 4B: Tumor growth over time. Tumor area estimated by multiplication of opposing perpendicular axes. Individual time points analyzed by one-way ANOVA (*p<0.05; **p<0.01) and survival analysis.

FIG. 5A: Nucleotide sequence (SEQ ID NO: 1) of Mip3α (mouse) and GP100 (human) in bacterial protein expression plasmid, pET-47b(+).

FIG. 5B: Amino acid sequence (SEQ ID NO: 2) expressed from nucleotide sequence (SEQ ID NO: 1) of Mip3α (mouse) and GP100 (human) in bacterial protein expression plasmid, pET-47b(+).

FIG. 6A: Nucleotide sequence (SEQ ID NO: 3) of (mouse) Mip3α-(human) GP100 sequence in mammalian protein expression plasmid, pCMVEa/b or VR1012.

FIG. 6B: Amino acid sequence (SEQ ID NO: 4) expressed from nucleotide sequence (SEQ ID NO: 3) (mouse)Mip3α-(human)GP100 sequence in mammalian protein expression plasmid, pCMVEa/b or VR1012.

FIG. 7A: Nucleotide sequence (SEQ ID NO: 5) of human Mip3α.

FIG. 7B: Amino acid sequence (SEQ ID NO: 6) expressed from nucleotide sequence (SEQ ID NO: 5) of human Mip3α.

FIG. 8: Tumor size reduction 17 days post-tumor induction in animals based on various treatment groups.

FIG. 9: Percent survival over days post-tumor induction in animals based on various treatment groups.

### DETAILED DESCRIPTION

In certain embodiments, multiple facets of the tumor system are simultaneously targeted. These treatments may have separate modes of action, and their combination may provide a synergistic action to dramatically improve patient treatment. αCTLA-4 may be combined with granulocyte-macrophage colony-stimulating factor (GM-CSF), which can increase overall survival while decreasing toxicity. In another embodiment, a recombinant adenovirus vaccine was given in combination with αPD-1 and α4-1BB (CD137), which elicited melanoma remission in mice. In certain embodiments, combination therapies including αCTLA-4 and αPD-1, have improved outcomes.

In a murine embodiment, a dendritic cell vaccine with GP100 antigen was combined with αIL-10. Certain embodiments may not utilize problematic virus vectors or expensive and technically demanding adoptive dendritic cell transfers. Further, the present invention is to provide a level of specific immunity to protect against development of recurrences or metastases, which would not be the case for combinations of antibody and chemotherapies.

The addition of therapeutic antibodies αIL-10 and αPD-1 to certain embodiments is to produce a synergistic interaction between a vaccine construct and countermeasures to address tumor-initiated immunosuppression and to modulate or completely suppress tumor growth and spread; thus, prolonging median survival by 47%. Combination treatments may include agents that affect multiple facets of tumorigenesis, shrinking primary tumors while building vaccination-induced immunity to eliminate metastases and prevent relapses.

In certain embodiments, tumor microenvironment immune parameters can act as non-invasive corollaries of protection in a combination therapy system. In certain embodiments, a melanoma model with IL-10 knockout mice on the C57Bl/6 background is utilized. In certain embodiments, the knockout mouse in combination with therapeutic vaccination of MIP-3α-GP100 to analyze local immune parameters can be utilized. The present invention provides, in part, a focus on levels of pro- and anti-inflammatory cytokines such as Interferon-γ, Tumor Necrosis Factor-α, Transforming Growth Factor-β, etc.; counts of different immune cell types, especially Tregs and CD8⁺ T-cells; and percentage of GP100-specific Tumor Infiltrating Lymphocytes (TILs).

The effect of immunological interventions on the tumor microenvironment can be characterized to identify corollaries of effective therapy. Using methods similar to those described in U.S. Pat. No. 8,557,248 B2, herein incorporated by reference, qRT-PCR can be used to identify alterations in the cytokine/chemokine environment within the tumor, as well as examine by flow cytometry the composition of immune cell populations within the tumor. Focus may be placed on an array of cytokines including, but not limited to IFN-γ, TNF-α, TGF-β, IL-2, IL-6, IL-10, and IL-17. Flow cytometry can be employed to quantify the presence of different immune cell types, especially regulatory T cells (Treg) and CD8+ T-cells, and percentage of GP100-specific Tumor Infiltrating Lymphocytes (TILs).

Certain embodiments can utilize melanoma combination therapy utilizing therapeutic antibodies and MIP-3α-antigen fusion vaccination. Some embodiments can utilize melanoma-associated antigens in the same plasmid or in separate plasmids fused to MIP-3α. Complimentary antibodies in addition to αIL-10 and αPD-1 can be added to the therapy to enhance protective effects. The synergistic efficacy of blocking other checkpoint inhibitors and activating effector T-cells can be improved using the embodiments.

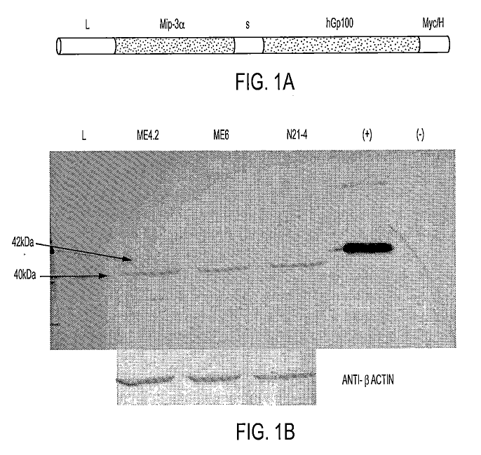

FIGS. 1A and 1B show a schematic of an embodiment of a vaccination construct and in vitro proof of cellular protein production. In certain embodiments, an insert is included in the pCMVeA/B plasmid, a pCMV backbone with minor modifications. DNA encoding the leader sequence for the heavily secreted mouse chemokine IP-10 (CXCL10) can be included at the 5′ end. DNA encoding the leader can be attached to DNA encoding full-length mouse MIP-3α chemokine followed by a short spacer region, DNA encoding amino acids (aa) 25-235 of the human gp100 protein, and DNA encoding standard myc and histidine tags at the 3′ end. As necessary, the present invention also allows for the absence of the IP-10 signal sequence and utilizes human MIP-3α versus the mouse MIP-3α. The invention also allows for removal of the myc tag as necessary. The primary immunodominant Class-I epitope can be conserved between mice and humans and may be included in the construct (DNA encoding aa 25-33). Plasmids can be transfected into the mammalian cell line 293T by Lipofectamine® procedure to confirm protein production in mammalian cells. Protein production can then be analyzed by western blot shown as shown in FIG. 1B. In FIG. 1B, arrows point to 40 kDa, the approximate size of the full construct, and 42 kDa. The band at 42 kDa is the same size as secreted protein in the supernatant (data not shown). Gp100 has many glycosylation sites. Gp100 also has natural cleavage sites, which explain the smaller bands. This provides evidence that sufficient protein can be produced and secreted in vivo after tissue transfection. Gp100 is representative of any melanoma antigen, specifically novel antigens that appear during disease progression. Cancers mutate as they grow and there is significant interest in targeting the neoantigens that appear during this process. While it would be difficult to provide a sequence for these neoantigens because they will be specific to each individual, targeting these is part of a new interest in personalized or precision medicine. Neoantigens are antigens discovered through “high throughput”, “next generation” or “advanced” sequencing techniques.

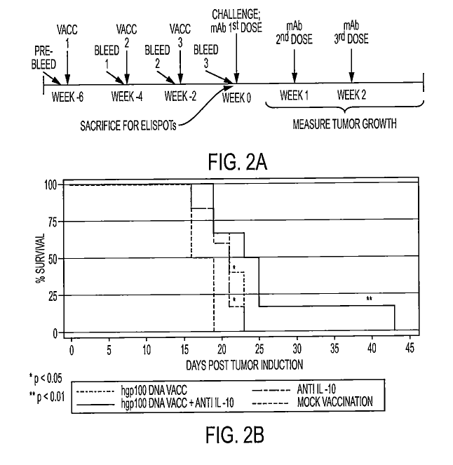

After in vitro analysis, the model can be tested in a prophylactic setting. The standard mouse model of melanoma in young female C57Bl/6 mice can be utilized. FIG. 2A shows an exemplary experimental schedule. Mice can be vaccinated by in vivo intra-muscular (i.m.) electroporation (BTX© ECM 830). Electroporation is also highly efficient for the introduction of foreign genes into tissue culture cells, especially mammalian cells. For example, it can be used in the process of producing knockout mice, as well as in tumor treatment, gene therapy, and cell-based therapy. One process of introducing foreign DNA into eukaryotic cells is known as transfection. Electroporation can also be highly effective for transfecting cells in suspension using electroporation cuvettes. Electroporation can be utilized on tissues in vivo, for in utero applications as well as in ovo transfection. Each vaccination can be administered in the shaved tibialis muscle and contain 50 μg plasmid. A plasmid can be purified by Qiagen© EndoFree® kits and may be analyzed by gel electrophoresis, restriction enzyme analysis, spectrophotemetry, and full insert sequencing. 150 μg per dose αIL-10 (clone JES5-2A5, BioXcell) can be given subcutaneously at challenge site(s). Tumors can be induced by subcutaneous injection with B16F10 melanoma cells in a mouse inner flank. Average tumor growth can be significantly delayed in vaccination groups, and the combination group can demonstrate improved results. Most importantly, the endpoint analysis (FIG. 2B) shows that αIL-10 by itself can significantly enhances survival, as does vaccine alone, and that embodiments combining the two can provide an unexpected effect leading to highly significant enhanced survival.

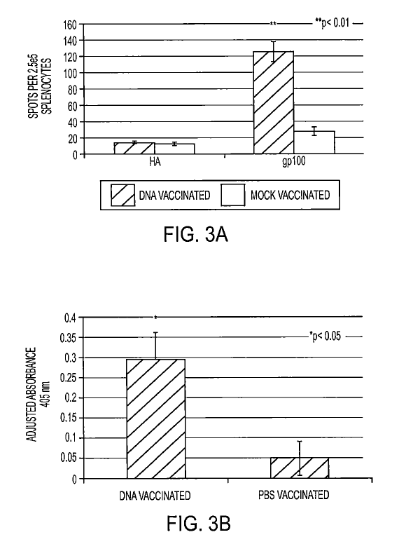

FIGS. 3A and 3B address the assessments of both cell-mediated and humoral arms of the adaptive immune system. ELISpot assays may be performed by standard lab protocol utilizing irradiated EL-4 syngeneic T-cells as antigen-presenting cells (APCs), loaded with either negative or gp100 peptides. Activation levels of isolated mouse splenocytes can be determined by incubating them with antigen-loaded APCs for the required amount of hours and measuring IFN-γ output using a capture assay. Certain embodiments can show a clear and strong systemic cell-mediated immune response against gp100. Humoral immunity can be assessed by an In Cell ELISA assay, briefly described in FIG. 3. A significant increase in anti-B16 antibodies can be seen in vaccinated mice. The various embodiments can further demonstrate the tolerance-breaking ability of the vaccination.

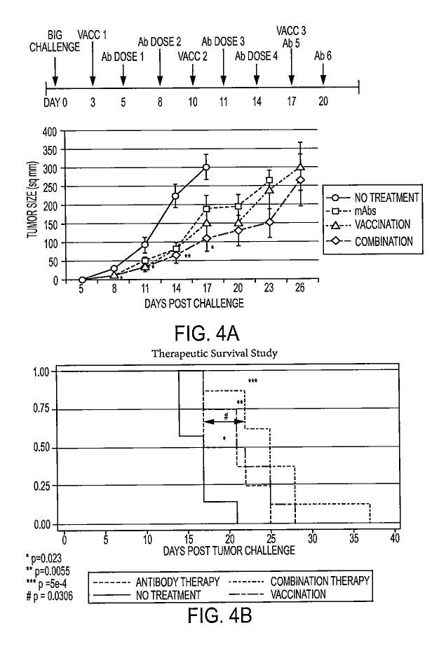

Certain embodiments can demonstrate efficacy in a clinical therapeutic model, where treatments do not begin until after tumor is induced. FIGS. 4A and 4B describe a therapeutic model, with FIG. 4A showing an embodiment of a treatment schedule. All other doses and details can be the same as FIGS. 2A and 2B, except αPD-1 (clones RMP1-14 and J43, BioXcell) can be given at a dose 250 μg intra-peritoneally (i.p.) in addition to αIL-10. Tumor size data shows that vaccine treatments, antibody therapy, and the combination of the two can delay growth significantly, with greatest delay in the combination group. The survival analysis is consistent with the tumor size data. Single treatments may significantly improve survival, and the combination provided highly significant survival improvement, and can enhance the responses to the individual treatments alone. The vaccine platform provides significant efficacy in a therapeutic model, and the efficacy is enhanced by the addition of immunomodulatory antibodies.

A mouse model is useful for studying the impact of immunomodulation on tumor progression and survival. In an effort to balance having sufficient tumor mass for analysis without extensive necrosis, the immune environment in tumors at different time points can be characterized. The optimal time point for tissue examination may differ for groups receiving different or no therapeutic interventions, but coverage of a range of time points should provide important insights into the kinetics and magnitude of the responses associated with different intervention strategies. The initial groups to be compared can be those described in FIGS. 4A and 4B, with the first tissue harvest occurring around Days 10-12, depending on the pattern of tumor growth in the individual experiment. This would allow for sufficient tumor mass for analysis as well as allowing for sufficient time to see an effect of the immunologic interventions. It may also be useful to pool tumors within groups to have sufficient tumor mass for analysis. In some cases, tumor bearing, untreated mice serve as controls. Correlations between outcomes and specific cytokine/chemokine and cell populations provides insights into what components of the immune response correspond with tumor control. As indicated, an array of cytokines can be evaluated, but specifically including IFN-γ, TNF-α, TGF-β, IL-2, IL-6, IL-10, and IL-17. Tissue for flow cytometric analysis is prepared as previously described in Luo, K., et al., Fusion of antigen to a dendritic cell targeting chemokine combined with adjuvant yields a malaria DNA vaccine with enhanced protective capabilities PLoS One, 2014. 9(3): p. e90413, which is incorporated herein by reference, with the addition of methods facilitating analysis of TILs by intracellular cytokine staining. Some emphasis is to be placed on quantitating and characterizing CD11c+vs. CD123+ dendritic cells, CD4+ Foxp3+ regulatory T cells, granzyme+ and IFN-γ+CD8+ T cells, and CD11b+, Gr1+ myeloid-derived suppressor cells. These analyses are to allow for the examination of the effects of our interventions on the balance between regulatory and effector immune functions. These outcomes can be statistically analyzed by ANOVA.

Having defined the local immune parameters associated with initial interventions, those interventions can be supplemented by using an additional antigen and additional antibodies (summarized in Table 1) to determine their impact on tumor growth, survival, and on the parameters outlined above. The impact of an additional antigen to the vaccine regimen may be incorporated into various embodiments. Utilizing standard bacterial cloning procedures, DNA encoding the clinically relevant antigen tryosinase-related protein 2 (TRP-2) can be inserted into a separate plasmid fused to MIP-3α, as was done for gp100 (FIGS. 1A and 1B). The construct can be confirmed and plasmid purified as noted previously. In addition to having two antigens incorporated into two separate plasmids, DNA encoding the two antigens, appropriately separated by spacer sequences, can be incorporated into a single plasmid, along with the other components of the vaccine platform. The efficacy of exposure to two antigens can initially be evaluated comparing the two-antigen vaccine with the same regimen using the gp100 and TRP-2 constructs alone. Initially, survival and tumor size parameters may determine which construct(s) would be beneficial for further studies. If the response to two antigens does not differ from the response to one, certain embodiments can incorporate two antigens. Immune editing, loss of antigens by the tumor due to immune selection pressure, can be more relevant in the extended time course of the clinical setting, as opposed to the rapid tumor time course in the mouse model. An advantage for a DNA immunization platform is the ability to alter vaccine antigens if patient tumors have been demonstrated to lose a particular targeted antigen or to target new antigens that appear as a result of mutations that occur in rapidly growing tumors.

Following alteration of a vaccine construct, different therapeutic antibodies can be tested for synergistic efficacy within this system, beginning with a αIL-10 and αPD1 regimen. For the clinical setting, it may be impractical to administer αIL-10 at the tumor site, especially in the context of metastatic disease. The use of systemic αIL-10 monoclonal antibody may demonstrate no significant toxicity following daily administration over a 21-day interval at a dose of 0.25 mg/kg. To evaluate the potential efficacy of systemic depletion of IL-10, tumor size and survival among three αIL-10 treatment groups may be compared: mice treated with the vaccine construct plus intravenous administration of αIL-10 at doses that range between those used in the human studies to doses that have been used previously in mice (5-250 μg/mouse), mice receiving the previously used local injections at the tumor site, and IL-10 knockout mice. Subsequent experiments will add α4-1BB (CD137), an antibody which acts as an agonist for CD8+ T cell activation and may synergistically inhibit tumor growth when combined with vaccine and αPD1. An embodiment incorporating this regimen can avoid the adverse events that are commonly associated in high frequency with αCTLA4, which can be used in combination with αPD1. The addition of αCTLA4 to the regimen can also be used in the clinical setting. Appropriate controls with and without the vaccine construct and different combinations of the antibodies can be included in all of the comparison studies.

The described regimens may produce complete remission, thus allowing further study to be performed on the mice two months after initial challenge. These mice can be challenged again in the opposite flank and/or intravenously to assess protection from relapse or metastases. These alterations to the protocol can lead to a therapy to greatly alter the course of clinical disease. All data can be statistically analyzed by ANOVA and log-rank tests, as described in preliminary results and as discussed above.

TABLE 1
Summary and rationale of treatment additions to be tested by
embodiments of a vaccine:

| Order of treatment additions to | |
|---|---|
| original vaccine platform | Rationale |
| Antigen TRP-2 | Multiple targets avoids immunoediting |
| Systemic αIL-10 | Expand protection observed with local |
| | application |
| α4-1BB | CD8+ T-cell coactivator known to |
| | synergize significantly with αPD-1 |
| αCTLA-4 | Checkpoint inhibitor known to |
| | synergize significantly with αPD-1 |

In one embodiment utilizing a therapy combining MIP-3α-antigen fusion DNA vaccines with immunomodulatory antibodies can have potent effects against melanoma in the mouse model and in human patients. Mechanistic immunological correlations can be utilized to fully assess an optimized therapy. An embodiment that induces established tumors to undergo remission by establishment of immunity to multiple antigens via vaccination and by reversal of the anti-immunity tumor microenvironment via a cocktail of immunomodulatory therapeutic antibodies can be utilized. This has great potential clinical impact, because the treatment could not only increase patient short-term outcomes, but could also help prevent metastases and long-term relapses.

The following is a table of adjuvants organized by class and with examples for the melanoma vaccine described herein. These adjuvants will aid in obtaining a high antibody concentration, including use of adjuvants with a protein formulation.

TABLE 2
Main adjuvant classes with representative examples:

| Class | Example |
|---|---|
| Delivery systems | Alum adjuvants, calcium phosphate, liposomes, |
| | virosomes, emulsions (e.g. MF59, montanides), virus-like |
| | particles, ISCOMS, etc |
| Immunopotentiators | Muramyl dipeptide (MDP and derivatives), |
| | monophosphoryl lipid A (MPL and its derivates), |
| | oligonucleotides such as polyinosinic:polycytidilic acid, |
| | saponins (QS-21, quils), chemokines and cytokines |
| Polymeric microsphere adjuvants | Biodegradable and biocompatible microspheres |
| | incorporating antigens of various types e.g. poly (DL- |
| | lactide-coglycolide) (DL-PLG), polyanhydrides, etc. |
| Carbohydrate based adjuvants | Complex carbohydrates of natural origin activating both |
| | humoral and cellular immune responses e.g. gamma- |
| | inulin, glucans, xylans, acemannan, etc. |
| Cytokines | A full-fledged adjuvant class enhancing cellular immune |
| | response through different mechanisms e.g. IFN-γ, |
| | IFN-α, IL-1, IL-6, IL-12 and GM-CSF. |
| Bacterial products | Cell wall lipopolysaccharide (LPS) or peptidoglycan |
| | products, trehalose dimycolate (TDM), MDP, MLP or |
| | their synthetic derivatives targeting mostly the TLRs. |

One embodiment of the present invention is the use of a melanoma vaccine construct described for the DNA vaccine but expressing the DNA in bacteria as a protein. It should be understood that the protein can also be expressed in yeast, insect cells or mammalian cells. One strength of this approach is this vaccine is readily adaptable to the appearance of new cancer antigens, termed neoantigens, which arise as a result of ongoing mutations of tumor genes. Therefore, the present invention includes a DNA vaccine framework into which a tumor antigen is inserted and the expression product can be recognized by the immune system. The approach of identifying neoantigens is described in Castle et al. (2012), Exploiting the Mutanome for Tumor Vaccination, Cancer Research; 72(5); 1081-91. While Castle et al. emphasizes neoantigen identification by CD8+ T cells, the present invention is adapted to recognize and identify new tumor proteins that are recognized by antibody and also CD4+ T cells with a separate screening.

As provided in more detail below, the present invention provides for standalone DNA vaccine constructs, standalone protein vaccine constructs, or even combinations of the two. In certain embodiments, the DNA vaccine can be the MIP-3α-melanoma-associated antigen fusion construct used in combination with anti-interleukin-10 (“anti-IL-10”). In other embodiments, the protein formulation of a vaccine construct can be the MIP-3α-melanoma-associated antigen fusion construct in combination with an adjuvant as described herein. The protein formulation of the vaccine construct can be the MIP-3α-melanoma-associated antigen fusion construct in combination with an adjuvant and/or anti-IL-10.

By way of example, several compositions and therapies were used to compare post-induction tumor size and survival of individuals post-tumor induction. This study utilized exclusively 6-8 week old female C57BL/6 mice ordered from Charles River Laboratories (Wilmington, Mass.). Mice were challenged in the left flank subcutaneously with a lethal dose (5×10⁴ cells) of B16F10 melanoma. Tumor size was recorded as square mm, representing length×width (opposing axes) measured by calipers every 1-3 days. The mice were kept in the study until one of the following occured: mouse death, tumor size eclipsing 20 mm in any direction, or extensive tumor necrosis and ulceration. Anti-IL-10 antibody 150 ug/injection; BioXcell JES5.2A51 was administered subcutaneously at the challenge/tumor site beginning day 5 post tumor challenge and continuing once every 3 days for a total of 6 doses. The vaccination plasmid extracted from E. coli using Qiagen® EndoFree® Plasmid Maxi and Giga Kits were used. DNA verified by gel electrophoresis, restriction enzyme analysis, Nanodrop® spectrophotometry, and full insert sequencing. The vaccine comprised solely of purified plasmid DNA encoding MIP-3α-gp100 fusion sequence in endotoxin-free PBS. Mock vaccinations were comprised of endotoxin-free PBS only. DNA injections were administered into the hind leg tibialis muscle. Immediately following injection, the muscle was pulsed using an ECM 830 Electro Square Porator (BTX Harvard Apparatus®) with the following parameters: 106V; 20 ms pulse length; 200 ms pulse interval; 8 total pulses. Vaccinations of 50 ug/dose delivered at days 3, 10, and 17 post tumor challenge. For survival studies, groups included 22-29 mice encompassing 3-4 independent experiments. For analysis of tumor size, groups included 22-47 mice across 4-9 independent experiments, and analysis of day 17 specifically included 22-35 mice per group across 4-6 independent experiments. Tumor size analyses were statistically tested by one-way anova with bonferonni correction. Mouse survival studies were statistically tested by the log-rank test. α<0.05.

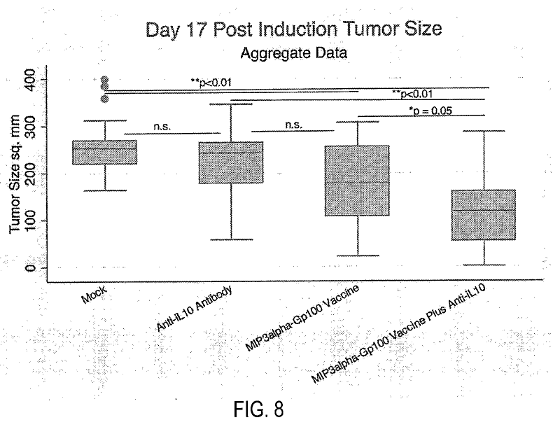

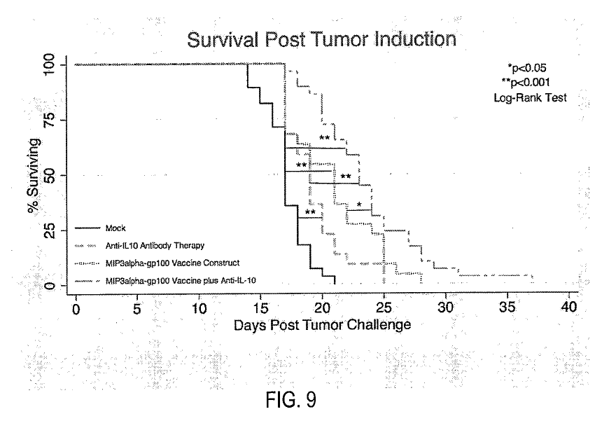

For the comparison, melanoma tumors were introduced to animals in the laboratory. The animals were divided into groups for control group (mock), anti-IL-10 treatment group, DNA MIP-3α-GP100 vaccine construct treatment group, and DNA MIP-3α-GP100 vaccine construct plus anti-IL-10 treatment group. As seen in FIG. 8, tumor size 17 days post tumor introduction was significantly different for the DNA MIP-3α-GP100 vaccine construct plus anti-IL-10 treatment group. The DNA MIP-3α-GP100 vaccine construct plus anti-IL-10 treatment group also showed a significant increase in percent survival of animals post-tumor induction as seen in FIG. 9. The DNA MIP-3α-GP100 vaccine construct plus anti-IL-10 treatment group had a significantly higher survival percentage 35 days post-tumor induction. Even the DNA MIP-3α-GP100 vaccine construct showed substantial tumor size reduction and post-induction survival. The data shows that the combination of DNA MIP-3α-GP100 vaccine construct plus anti-IL-10 is an unexpected and effective vaccine therapy against melanoma.

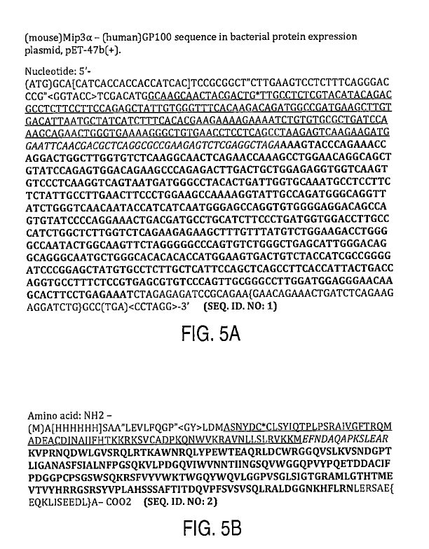

In FIG. 5A, a melanoma DNA vaccine construct of the present invention is provided that comprises (mouse) MIP-3α-(human) GP100 sequence (SEQ ID NO: 1) in bacterial protein expression plasmid, pET-47(+). As discussed herein, the protein expressed by this construct can be used alone, or in a separate embodiment, used in combination with an adjuvant. Another embodiment of the present invention is the formation and use of the protein formulation of the MIP-3α-vaccine antigen fusion construct in combination with an adjuvant and anti-IL-10. Another embodiment would be the formation and use of the protein formulation of the MIP-3α-vaccine antigen fusion construct in combination with the anti-IL-10. In certain embodiments, the protein construct can be used in combination with other therapeutic compositions. Embodiments of the present invention include:

Nucleotide:
(SEQ ID NO: 1)
5′-(ATG)GCA[CATCACCACCACCATCAC]TCCGCGGCT“CTTGAAGT
CCTCTTTCAGGGACCCG”<GGTACC>TCGACATGGCAAGCAACTACGAC
TG*TTGCCTCTCGTACATACAGACGCCTCTTCCTTCCAGAGCTATTGTG
GGTTTCACAAGACAGATGGCCGATGAAGCTTGTGACATTAATGCTATCA
TCTTTCACACGAAGAAAAGAAAATCTGTGTGCGCTGATCCAAAGCAGAA
CTGGGTGAAAAGGGCTGTGAACCTCCTCAGCCTAAGAGTCAAGAAGATG
GAATTCAACGACGCTCAGGCGCCGAAGAGTCTCGAGGCTAGAAAAGTAC
CCAGAAACCAGGACTGGCTTGGTGTCTCAAGGCAACTCAGAACCAAAGC
CTGGAACAGGCAGCTGTATCCAGAGTGGACAGAAGCCCAGAGACTTGAC
TGCTGGAGAGGTGGTCAAGTGTCCCTCAAGGTCAGTAATGATGGGCCTA
CACTGATTGGTGCAAATGCCTCCTTCTCTATTGCCTTGAACTTCCCTGG
AAGCCAAAAGGTATTGCCAGATGGGCAGGTTATCTGGGTCAACAATACC
ATCATCAATGGGAGCCAGGTGTGGGGAGGACAGCCAGTGTATCCCCAGG
AAACTGACGATGCCTGCATCTTCCCTGATGGTGGACCTTGCCCATCTGG
CTCTTGGTCTCAGAAGAGAAGCTTTGTTTATGTCTGGAAGACCTGGGGC
CAATACTGGCAAGTTCTAGGGGGCCCAGTGTCTGGGCTGAGCATTGGGA
CAGGCAGGGCAATGCTGGGCACACACACCATGGAAGTGACTGTCTACCA
TCGCCGGGGATCCCGGAGCTATGTGCCTCTTGCTCATTCCAGCTCAGCC
TTCACCATTACTGACCAGGTGCCTTTCTCCGTGAGCGTGTCCCAGTTGC
GGGCCTTGGATGGAGGGAACAAGCACTTCCTGAGAAATTCTAGAGAGAT
CCGCAGAA{GAACAGAAACTGATCTCAGAAGAGGATCTG}GCC(TGA)<
CCTAGG>-3′.

Internal annotations are described herein below.

In FIG. 5B, a melanoma protein vaccine construct of the present invention is provided that comprises (mouse) MIP-3α-(human) GP100 sequence (SEQ ID NO: 2) as expressed from a bacterial protein expression plasmid, pET-47(+). As discussed herein, this construct can be used alone or in combination with an adjuvant. In certain embodiments, the construct can be used in combination with other therapeutic compositions. Embodiments of the present invention include:

Amino acid:
(SEQ ID NO: 2)
NH2-(M)A[HHHHHH]SSA“LEVLFQGP”<GY>LDMASNYDC*CLSYIQ
TPLPSRAIVGFTRQMADEACDINAIIFHTKKRKSVCADPKQNWVKRAVN
LLSLRVKKMEFNDAQAPKSLEARKVPRNQDWLGVSRQLRTKAWNRQLYP
EWTEAQRLDCWRGGQVSLKVSNDGPTLIGANASFSIALNFPGSQKVLPD
GQVIWVNNTIINGSQVWGGQPVYPQETDDACIFPDGGPCPSGSWSQKRS
FVYVWKTWGQYWQVLGGPVSGLSIGTGRAMLGTHTMEVTVYHRRGSRSY
VPLAHSSSAFTITDQVPFSVSVSQLRALDGGNKHFLRNLERSAE{EQKL
ISEEDL}ACOO2.

Internal annotations are described herein below.

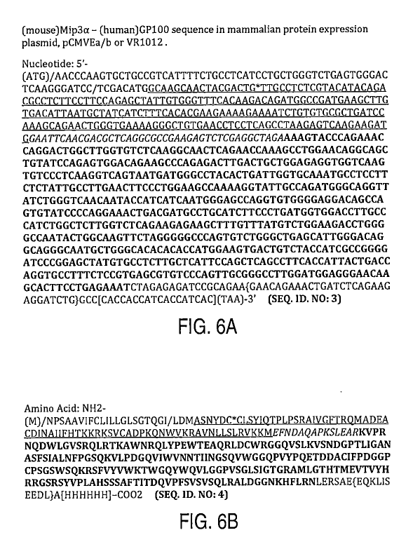

In FIG. 6A, a melanoma DNA vaccine construct of the present invention is provided that comprises (mouse) MIP-3α-(human) GP100 sequence (SEQ ID NO: 3) in mammalian protein expression plasmid, pCMVeA/B or VR1012. As discussed herein, this construct can be used alone or in combination with anti-IL-10 and/or an adjuvant. In certain embodiments, the construct can be used in combination with other therapeutic compositions. Embodiments of the present invention include:

Nucleotide:
(SEQ ID NO: 3)
5′-(ATG)/AACCCAAGTGCTGCCGTCATTTTCTGCCTCATCCTGCTGG
GTCTGAGTGGGACTCAAGGGATCC/TCGACATGGCAAGCAACTACGACT
G*TTGCCTCTCGTACATACAGACGCCTCTTCCTTCCAGAGCTATTGTGG
GTTTCACAAGACAGATGGCCGATGAAGCTTGTGACATTAATGCTATCAT
CTTTCACACGAAGAAAAGAAAATCTGTGTGCGCTGATCCAAAGCAGAAC
TGGGTGAAAAGGGCTGTGAACCTCCTCAGCCTAAGAGTCAAGAAGATGG
AATTCAACGACGCTCAGGCGCCGAAGAGTCTCGAGGCTAGAAAAGTACC
CAGAAACCAGGACTGGCTTGGTGTCTCAAGGCAACTCAGAACCAAAGCC
TGGAACAGGCAGCTGTATCCAGAGTGGACAGAAGCCCAGAGACTTGACT
GCTGGAGAGGTGGTCAAGTGTCCCTCAAGGTCAGTAATGATGGGCCTAC
ACTGATTGGTGCAAATGCCTCCTTCTCTATTGCCTTGAACTTCCCTGGA
AGCCAAAAGGTATTGCCAGATGGGCAGGTTATCTGGGTCAACAATACCA
TCATCAATGGGAGCCAGGTGTGGGGAGGACAGCCAGTGTATCCCCAGGA
AACTGACGATGCCTGCATCTTCCCTGATGGTGGACCTTGCCCATCTGGC
TCTTGGTCTCAGAAGAGAAGCTTTGTTTATGTCTGGAAGACCTGGGGCC
AATACTGGCAAGTTCTAGGGGGCCCAGTGTCTGGGCTGAGCATTGGGAC
AGGCAGGGCAATGCTGGGCACACACACCATGGAAGTGACTGTCTACCAT
CGCCGGGGATCCCGGAGCTATGTGCCTCTTGCTCATTCCAGCTCAGCCT
TCACCATTACTGACCAGGTGCCTTTCTCCGTGAGCGTGTCCCAGTTGCG
GGCCTTGGATGGAGGGAACAAGCACTTCCTGAGAAATCTAGAGAGATCC
GCAGAA{GAACAGAAACTGATCTCAGAAGAGGATCTG}GCC[CACCACC
ATCACCATCAC](TAA)-3′.

Internal annotations are described herein below.

In FIG. 6B, a melanoma protein vaccine construct of the present invention is provided that comprises (mouse) MIP-3α-(human) GP100 sequence (SEQ ID NO: 4) expressed from SEQ ID NO: 3 from a mammalian protein expression plasmid, pCMVeA/B or VR1012. As discussed herein, this construct can be used alone or in combination with an adjuvant. In certain embodiments, the construct can be used in combination with other therapeutic compositions. Embodiments of the present invention include:

Amino Acid:
(SEQ ID NO: 4)
NH2-(M)/NPSAAVIFCLILLGLSGTQGI/LDMASNYDC*CLSYIQTPL
PSRAIVGFTRQMADEACDINAIIFHTKKRKSVCADPKQNWVKRAVNLLS
LRVKKMEFNDAQAPKSLEARKVPRNQDWLGVSRQLRTKAWNRQLYPEWT
EAQRLDCWRGGQVSLKVSNDGPTLIGANASFSIALNFPGSQKVLPDGQV
IWVNNTIINGSQVWGGQPVYPQETDDACIFPDGGPCPSGSWSQKRSFVY
VWKTWGQYWQVLGGPVSGLSIGTGRAMLGTHTMEVTVYHRRGSRSYVPL
AHSSSAFTITDQVPFSVSVSQLRALDGGNKHFLRNLERSAE{EQKLISE
EDL}A[HHHHHH](.)-COO2.

Internal annotations are described herein below.

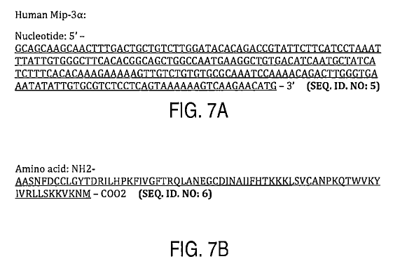

In FIG. 7A, a melanoma DNA vaccine construct of the present invention is provided that comprises (human) MIP-3α-(human) GP100 sequence (SEQ ID NO: 5), wherein human MIP-3α is utilized in place of mouse MIP-3α. As discussed herein, this construct can be used alone or in combination with an adjuvant. In certain embodiments, the construct can be used in combination with other therapeutic compositions. Embodiments of the present invention include:

Nucleotide:
(SEQ ID NO: 5)
5′-GCAGCAAGCAACTTTGACTGCTGTCTTGGATACACAGACCGTATTC
TTCATCCTAAATTTATTGTGGGCTTCACACGGCAGCTGGCCAATGAAGG
CTGTGACATCAATGCTATCATCTTTCACACAAAGAAAAAGTTGTCTGTG
TGCGCAAATCCAAAACAGACTTGGGTGAAATATATTGTGCGTCTCCTCA
GTAAAAAAGTCAAGAACATG-3′.

Internal annotations are described herein below.

In FIG. 7B, a melanoma protein vaccine construct of the present invention is provided that comprises (human) MIP-3α-(human) GP100 sequence (SEQ ID NO: 6), wherein human MIP-3α is utilized in place of mouse MIP-3α. As discussed herein, this construct can be used alone or in combination with an adjuvant. In certain embodiments, the construct can be used in combination with other therapeutic compositions. Embodiments of the present invention include:

Amino acid:
(SEQ ID NO: 6)
NH2-AASNFDCCLGYTDRILHPKFIVGFTRQLANEGCDINAIIFHTKK
KLSVCANPKQTWVKYIVRLLSKKVKNM-COO2.

Internal annotations are described herein below.

As shown in SEQ ID NOs: 1-6 above, there are various internal annotations, which are described in the following key:

Key:
(Parentheses): Start and Stop codons
[Brackets]: Histidine Tag. This tag is used for in vitro purification and/or identification.
“Quotes”: HRV 3C Protease sequence. A part of the pET-47b(+) plasmid, this sequence allows you to add a protease that will selectively cut off the histidine tag so that it will not be in your final protein destined for therapy.
<Wedges>: Restriction sites utilized in pET-47b(+) plasmid. 5′ end KpnI; 3′ end AvrII. Our vaccine sequence was inserted into the plasmid utilizing these restriction sites.
Underlined: Mip-3α sequence
*Asterisked: Mutated in control plasmid with defective Mip-3α. Guanine changed to cytosine, changing the cysteine amino acid to serine. This abrogates the function of Mip-3α without changing the length of the construct.
Italics: Spacer sequence to allow Mip-3α and gp100 to fold correctly
Bold: Human gp100, amino acids 25-235
{Braces}: c-myc tag. This is a standard in vitro tag that allows for easy and specific detection of protein in western blots, elisas, and other antibody-based assays.
/Slashes/: Mouse IP-10 Leader sequence. IP-10 is a secreted mouse cytokine. However, this is only the leader sequence from that gene that contains the necessary peptide motifs for cellular excretion in eukaryotic and especially mammalian based systems. It is not present in constructs utilized for bacterial protein production.

As discussed herein, the present invention provides for standalone DNA vaccine constructs, standalone protein vaccine constructs, and combinations thereof. As provided herein, the sequences of certain DNA and protein vaccine construct embodiments are provided. While certain methods of expressing the desired vaccine constructs are disclosed, other methods for expressing the desired vaccine constructs are known. Delivery methods for the DNA and protein vaccine constructs are known. These include plasmid DNA delivery methods of: parenteral delivery (e.g., injection, gene gun, pneumatic (jet) injection); topical application; and cytofectin-mediated delivery.

Administration of the protein vaccine construct of the present invention to an individual in need of melanoma therapeutic care is expected to yield high concentrations of antibodies in the subject. The high concentrations of antibodies created by the protein vaccine construct of the current invention can be used in combination with T cells to develop a chimeric antigen receptor type (CAR T) system. A recently described technology employed for cancer immunotherapy uses T cells carrying antibodies on their surface to target antigens on tumor cells (Prapa, et al. (2015), A novel anti-GD2/4-1BB chimeric antigen receptor triggers neuroblastoma cell killing. Oncotarget 6: 24884-24894; and Kudo, et al. (2014), T lymphocytes expressing a CD16 signaling receptor exert antibody-dependent cancer cell killing. Cancer research 74: 93-103). Essentially, T cells obtained from patients are engineered to express receptors for antibody on their surface. Termed “Antibody-Coupled T-cell Receptor (ACTR) technology”, this technology relies on an engineered T-cell component that can bind antibodies and use them to target the T-cells. When modified T-cells are put back into the patient, they can be targeted to attack tumors by co-administering cancer-specific antibodies. Patents covering the ACTR concept have been filed by St. Jude Children's Research Hospital and the National University of Singapore (U.S. Pat. No. 8,399,645).

The success of this technology is dependent on establishing high concentrations of antibody specific for a tumor antigen. This is applicable to the vaccine constructs of the present invention due to the high levels of antibodies resulting from administration of the vaccine constructs of this invention. While monoclonal antibodies have been used initially in studies of this technology, the ability to rapidly elicit high concentrations of antibodies to antigens for which monoclonal antibodies are not available and particularly for neoantigens that appear as tumor cells mutate would greatly enhance the potential efficacy of this approach. The ability of the MIP-3α vaccine platform to be rapidly modified to express antigens of interest and to elicit remarkably high concentrations of specific antibody should enhance the breadth of activity and ultimately the efficacy of this ACTR technology. An individual could be immunized with a vaccine platform/construct expressing the antigens of interest to be followed by infusion of the ACTR engineered T lymphocytes.

A number of embodiments of the invention have been described. Nevertheless, it will understood that various modifications may be made without departing from the spirit and scope of the invention. Accordingly, other embodiments are within the scope of the following claims.

1. A composition for treating melanoma comprising:
a melanoma vaccine construct comprising:
a chemokine macrophage inflammatory protein 3α (MIP-3α); and
a melanoma-associated antigen;
wherein the MIP-3α is fused to the melanoma-associated antigen,
and wherein the MIP-3α targets a nascent protein to immature dendritic cells (iDCs) that impact development of an adaptive immune response.

2. The composition of claim 1 wherein the melanoma-associated antigen is GP100.

3. The composition of claim 1 further comprising at least one of αCTLA-4, granulocyte-macrophage colony-stimulating factor (GM-CSF), αPD-1, α4-1BB (CD137), αIL-10 or combinations thereof.

4. The composition of claim 2 further comprising at least one of αCTLA-4, granulocyte-macrophage colony-stimulating factor (GM-CSF), αPD-1, α4-1BB (CD137), αIL-10 or combinations thereof.

5. The composition of claim 1, further comprising anti-Interleukin-10 (“anti-IL-10”).

6. The composition of claim 5, wherein the MIP-3α is human and the melanoma-associated antigen is human.

7. The composition of claim 6, wherein the melanoma-associated antigen is human GP100.

8. The composition of claim 1, wherein the MIP-3α and melanoma-associated antigen are in a mammalian protein expression plasmid.

9. The composition of claim 8, wherein the plasmid is pCMVeA/B or VR1012.

10. The composition of claim 8, wherein the MIP-3α is mouse and the melanoma-associated antigen is human.

11. The composition of claim 1, wherein the MIP-3α and melanoma-associated antigen are an expressed protein from a bacterial protein expression plasmid.

12. The composition of claim 1, wherein the MIP-3α and the melanoma-associated antigen are an expressed protein from a mammalian protein expression plasmid.

13. The composition of claim 1, wherein the MIP-3α is human.

14. The composition of claim 8, wherein an insert is included in the pCMVeA/B.

15. The composition of claim 8, wherein a leader sequence of IP-10 is included at the 5′ end, wherein said leader sequence leader is attached to full-length mouse MIP-3α chemokine followed by a short spacer region, amino acids (aa) 25-235 of human GP100 protein, and standard myc and histidine tags at the 3′ end.

16. The composition of claim 1 further comprising at least one adjuvant selected from the following classes of adjuvants: delivery system adjuvants; immunopotentiators; polymeric microsphere adjuvants; carbohydrate based adjuvants; cytokines; and bacterial products.

17. A method of treating melanoma comprising:
combining MIP-3α-antigen fusion DNA vaccines with immunomodulatory antibodies to form a therapeutic combination effective against melanoma; and
administering the therapeutic combination in an amount effective to treat melanoma.

18. A melanoma vaccine comprising SEQ ID NO: 2, SEQ ID NO: 4 or SEQ ID NO: 6, wherein the melanoma vaccine is formulated to effectively treat melanoma.

19. A melanoma vaccine comprising SEQ ID NO: 1, SEQ ID NO: 3 or SEQ ID NO: 5, wherein the melanoma vaccine is formulated to effectively treat melanoma.

20. The melanoma vaccine of claim 19 in combination with at least one adjuvant selected from the following classes of adjuvants: delivery system adjuvants; immunopotentiators; polymeric microsphere adjuvants; carbohydrate based adjuvants; cytokines; and bacterial products.


SEQUENCE LISTING (6 sequences)

SEQ ID NO: 1 - DNA, 1017 bp, Artificial Sequence
  Description of Artificial Sequence Synthetic polynucleotide
atggcacatcaccaccaccatcactccgcggctcttgaagtcctctttcagggacccgggtacctcgacatggcaagcaactacgactgttgcctctcgtacatacagacgcctcttccttccagagctattgtgggtttcacaagacagatggccgatgaagcttgtgacattaatgctatcatctttcacacgaagaaaagaaaatctgtgtgcgctgatccaaagcagaactgggtgaaaagggctgtgaacctcctcagcctaagagtcaagaagatggaattcaacgacgctcaggcgccgaagagtctcgaggctagaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactgaccaggtgcctttctccgtgagcgtgtcccagttgcgggccttggatggagggaacaagcacttcctgagaaatctagagagatccgcagaagaacagaaactgatctcagaagaggatctggcctgacctagg

SEQ ID NO: 2 - protein, 336 aa, Artificial Sequence
  Description of Artificial Sequence Synthetic polypeptide
MAHHHHHHSAALEVLFQGPGYLDMASNYDCCLSYIQTPLPSRAIVGFTRQMADEACDINAIIFHTKKRKSVCADPKQNWVKRAVNLLSLRVKKMEFNDAQAPKSLEARKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNLERSAEEQKLISEEDLA

SEQ ID NO: 3 - DNA, 1032 bp, Artificial Sequence
  Description of Artificial Sequence Synthetic polynucleotide
atgaacccaagtgctgccgtcattttctgcctcatcctgctgggtctgagtgggactcaagggatcctcgacatggcaagcaactacgactgttgcctctcgtacatacagacgcctcttccttccagagctattgtgggtttcacaagacagatggccgatgaagcttgtgacattaatgctatcatctttcacacgaagaaaagaaaatctgtgtgcgctgatccaaagcagaactgggtgaaaagggctgtgaacctcctcagcctaagagtcaagaagatggaattcaacgacgctcaggcgccgaagagtctcgaggctagaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactgaccaggtgcctttctccgtgagcgtgtcccagttgcgggccttggatggagggaacaagcacttcctgagaaatctagagagatccgcagaagaacagaaactgatctcagaagaggatctggcccaccaccatcaccatcactaa

SEQ ID NO: 4 - protein, 343 aa, Artificial Sequence
  Description of Artificial Sequence Synthetic polypeptide
MNPSAAVIFCLILLGLSGTQGILDMASNYDCCLSYIQTPLPSRAIVGFTRQMADEACDINAIIFHTKKRKSVCADPKQNWVKRAVNLLSLRVKKMEFNDAQAPKSLEARKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNLERSAEEQKLISEEDLAHHHHHH

SEQ ID NO: 5 - DNA, 213 bp, Homo sapiens
gcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatg

SEQ ID NO: 6 - protein, 71 aa, Homo sapiens
AASNFDCCLGYTDRILHPKFIVGFTRQLANEGCDINAIIFHTKKKLSVCANPKQTWVKYIVRLLSKKVKNM